(19) United States
(12) Patent Application Publication
(10) Pub. No.: US 2025/0235476 A1
(43) Pub. Date: Jul. 24, 2025

(54) METHODS AND COMPOSITIONS THAT ARE ALTERNATIVES TO ANTIBIOTICS

(71) Applicant: PPC PHARMACEUTICALS, LLC, Canonsburg, PA (US)

(72) Inventor: Sarath Malepati, Canonsburg, PA (US)

(21) Appl. No.: 19/175,236

(22) Filed: Apr. 10, 2025

Related U.S. Application Data
(63) Continuation of application No. PCT/US23/76825, filed on Oct. 13, 2023.
(60) Provisional application No. 63/379,410, filed on Oct. 13, 2022.

(51) Int. Cl. A61K 33/30; A61K 31/375; A61K 31/59; A61K 36/28; A61P 31/12

(52) U.S. Cl. CPC A61K 33/30; A61K 31/375; A61K 31/59; A61K 36/28; A61P 31/12

(57) ABSTRACT

A method of treatment for upper respiratory infections, where the treatment does not include use of antibiotic(s). Use of a nanoparticle delivery system and dose schedule can support the immune system, without the use of antibiotic(s). The nanoparticle delivery system includes Echinacea purpurea, zinc, and vitamin C. The nanoparticle delivery system can also include vitamin D.

### CROSS-REFERENCE TO RELATED APPLICATIONS

This application claims priority to U.S. Provisional Application 63/379,410, filed on Oct. 13, 2022; which is herein incorporated by reference in its entirety.

### FIELD

Over-the-counter medications and supplements are used with an intention to improve health, support the immune system, and avoid or shorten viral illnesses such as the common cold and other upper respiratory infections.

### BACKGROUND

Use of antibiotics is not risk-free. Antibiotics can often be misused to have serious health implications. The antibiotics can actually cause harm, for example in the form of eliminating normal bacteria that regulate critical physiological functions. One of the most common reasons that patients are prescribed antibiotics in a primary care setting is for upper respiratory infections (URIs). It is understood that URIs are the most common infectious disease among humans, and greater than 75% of URIs are not in fact caused by bacteria, but rather viruses. Antibiotics have no effect on viruses. Rapid diagnostic tests assessing whether an infection is bacterial or not are generally limited to very specific bacterial strains and can be limited by their accuracy as well as user error. Formal bacterial culture via nasal or throat swab to assess bacterial growth takes 48-72 hours for the results. By the time a throat culture can be finalized, the infection may spread even further with serious consequences for the patient. This presents a better-to-be-safe-than-sorry situation for the healthcare provider. Thus, the diagnostic limitations coupled with patient pressure for a tangible solution from the healthcare provider are the primary drivers of inappropriate antibiotic prescriptions for viral illnesses. Consequently, the great majority of people taking antibiotics for URIs are receiving them unnecessarily while their actual disease is not being treated. Inappropriate antibiotic prescription for respiratory tract indications is most prominent in adult patients. In one study, 46% of antibiotics written for respiratory tract infections were not indicated based on clinical guidelines. The over-use of antibiotics can also lead to further health complications for the individual; as well as for the population as a whole. As another example, inappropriate antibiotic use can select for multi-drug resistant organisms, colloquially called “super bugs,” that cannot be eradicated by conventional methods. These “super bugs” pose a serious and growing worldwide health concern. Despite these dangers, many patients with URIs continue to come to their physicians and health care providers requesting antibiotics that cannot and should not be used to treat viral infections.

### SUMMARY

In some aspects, the techniques described herein relate to a method of treating a viral infection, including: obtaining an orally deliverable system, wherein the orally deliverable system includes: a powder form of Echinacea purpurea, zinc, and vitamin C, wherein the powder form of Echinacea purpurea is from 1100 mg to 2200 mg, wherein the zinc is from 50 mg to 100 mg, and wherein the vitamin C is from 500 mg to 1000 mg; and orally ingesting the orally deliverable system.

In some aspects, the techniques described herein relate to a method, wherein the orally deliverable system further includes: vitamin D, wherein the vitamin D is from 50 mcg to 100 mcg.

In some aspects, the techniques described herein relate to a method of treating symptoms of an upper respiratory infection, including: obtaining an orally deliverable system, wherein the orally deliverable system includes: a powder form of Echinacea purpurea, zinc, and vitamin C, wherein the powder form of Echinacea purpurea is from 1100 mg to 2200 mg, wherein the zinc is from 50 mg to 100 mg, and wherein the vitamin C is from 500 mg to 1000 mg; and orally ingesting the orally deliverable system.

In some aspects, the techniques described herein relate to a method, wherein the orally deliverable system further includes: vitamin D, wherein the vitamin D is from 50 mcg to 100 mcg.

In some aspects, the techniques described herein relate to methods of treatment, wherein the method does not include using antibiotics.

In some aspects, the techniques described herein relate to methods of treatment, wherein a Day To Recovery (DTR) time is less than about 6 days.

In some aspects, the techniques described herein relate to methods of treatment, wherein the zinc is zinc acetate.

In some aspects, the techniques described herein relate to methods of treatment, wherein the zinc is an ionizable form of zinc.

In some aspects, the techniques described herein relate to methods of treatment, wherein the method does not include using antibiotics, DTR time is less than about 6 days, and zinc is zinc acetate.

In some aspects, the techniques described herein relate to methods of treatment, wherein the method does not include using antibiotics, DTR time is less than about 6 days, and zinc is an ionizable form of zinc.

### BRIEF DESCRIPTION OF THE DRAWINGS

FIG. 1 is a visual representation of the dosing scheme over the five-day treatment period, according to some embodiments.

FIG. 2 shows a Kernel density estimate (KDE) plot graph of the study results, according to some embodiments.

FIG. 3 shows a Kaplan-Meier (KM) Plot for Days To Recovery (DTR) Score of the study results, according to some embodiments.

FIG. 4 shows a KDE plot of Symptom Severity During Recovery (SSDR) Scores, according to some embodiments.

### DETAILED DESCRIPTION

Embodiments disclosed herein include a composition of materials, such as for example, Echinacea, vitamin C, and zinc. According to some embodiments, the composition also includes vitamin D.

According to some embodiments, a targeted delivery system is packaged in a preferred dose and dose schedule that optimizes their effectiveness. Some embodiments can also include honey and/or a nanoparticle delivery system to further increase the efficacy of the medications and supplements.

According to some embodiments, methods for treating a viral infection, such as for example URIs, include obtaining an orally deliverable system according to at least one of the embodiments disclosed herein, and orally ingesting the orally deliverable system. According to some embodiments, the orally deliverable system includes a powder form of Echinacea purpurea, zinc, and vitamin C. According to some embodiments, the orally deliverable system includes 1100 mg to 2200 mg of the powder form of Echinacea purpurea. According to some embodiments, the orally deliverable system includes 50 mg to 100 mg of zinc. According to some embodiments, the orally deliverable system includes 500 mg to 1000 mg of vitamin C. According to some embodiments, the orally deliverable system includes 50 mcg to 100 mcg of vitamin D. According to some embodiments, the orally deliverable system includes 1100 mg to 2200 mg of the powder form of Echinacea purpurea, 50 mg to 100 mg of zinc, and 500 mg to 1000 mg of vitamin C. According to some embodiments, the orally deliverable system includes 1100 mg to 2200 mg of the powder form of Echinacea purpurea, 50 mg to 100 mg of zinc, 500 mg to 1000 mg of vitamin C, and 50 mcg to 100 mcg of vitamin D.

The effectiveness of treating infections, in particular URIs, has been studied and the data showing the effectiveness is shown below and in the accompanied drawings.

An initial randomized double-blind placebo-controlled clinical trial has been performed to evaluate the utility of various embodiments of the compositions disclosed herein. In an example, the term “EZC” (also called “EZC Pak”) is used to describe an embodiment of the composition, which includes a combination of Echinacea, zinc, and vitamin C. EZC does not include any antibiotics or vitamin D. The term “EZCD” (also called “EZC Pak+Vitamin D”) is used to describe another embodiment of the composition, which includes a combination of Echinacea, zinc, vitamin C, and vitamin D. EZCD does not include any antibiotics. In some embodiments, the evaluation has been in the management of outpatient URIs. The evaluation of the efficacy of EZC and EZCD on the duration of illness and symptom severity of non-specific URIs (as an alternative to antibiotics) has been performed. A secondary analysis was carried out on patient satisfaction with using EZC and EZCD.

### SUMMARY OF THE TRIALS

Methods: A total of 360 patients across the United States were enrolled and randomized in a double-blind manner across two intervention groups, EZC, EZCD, and one placebo group. The study was conducted utilizing a smartphone-based app to screen, enroll and capture study data of the participants. Once a study participant reported the first symptoms of a URI, they were advised to take the intervention as directed and complete the daily symptom survey score until their symptoms resolved.

Results: The average EZC Pak participant recovered 1.39 days faster than placebo (p=0.017) than the average placebo participant. The average EZC Pak participant reported a 17.43% lower symptom severity score versus placebo (p=0.029). EZC Pak users reported 2.9 times higher patient satisfaction versus users of the placebo (p=0.012). The addition of vitamin D during this acute phase of illness neither benefited nor harmed illness duration or symptom severity.

Conclusions: The findings support the potential use of EZC as a viable alternative to patient request for antibiotics when none are deemed clinically necessary at the time of initial clinical presentation. The decision to replete vitamin D in the acute phase of URI is an individualized decision left to the patient and their clinician. EZC may play a critical role in improving outpatient URI management and antibiotic stewardship.

Vitamins, minerals, and herbs to support the immune system's clearance of infections have lacked uniform standards in preparation, formulation, potency, and actual usage. It is believed that the dosages necessary to confer therapeutic benefit demonstrated in supportive studies may be much higher than the dosages found in standard products commonly found over the counter in pharmacies and drug stores. A key advantage of the potential use of vitamin, mineral, and herbal preparations as a tool in URI management is the reduced risk of antimicrobial resistance, reduced exposure to the potential side effects of drugs, and the long-term preservation of antibiotic and antimicrobial efficacy when they are critically needed.

In the case of the Western herb Echinacea, the strongest data for its potential use in the treatment of URIs is likely in the form of Echinacea purpurea. In the case of the mineral zinc, the greatest potential benefit of its usage in the management of URIs may be in ionizable forms of zinc, such as zinc acetate. The utility of vitamin C supplementation in either the prevention or treatment of URIs has yielded mixed results in randomized clinical trials (RCTs), but may provide more benefit in the prevention of URIs in patients doing heavy exercise and undergoing similar short term physical stress.

There has been increased interest in the potential role of vitamin D in URI management during the COVID-19 pandemic. While data is mixed, some current data shows there may be potential benefit in vitamin D supplementation in reducing the incidence of URIs. However, a study evaluating early, acute repletion of vitamin D with high enteral bolus during active COVID-19 illness in intensive care unit patients did not show benefit in reducing 90-day all-cause mortality.

While there are a number of studies evaluating the use of individual vitamins, minerals, and herbs in the management of URIs, RCTs evaluating the potential role of combinations of herbs, vitamins, and minerals that individually have demonstrated data supporting their individual usage remain limited.

Disclosed herein are methods of using EZC, a 5-day dose pack of Echinacea purpurea, zinc acetate, and vitamin C to reduce the duration of illness and symptom severity in non-specific upper respiratory infection. Adding vitamin D to the base dose pack of Echinacea purpurea, zinc acetate, and vitamin C was also carried out.

### Methods

A total of 360 individuals, male or female over 18 years old, were enrolled by a third-party clinical research organization (CRO), to participate in the study. The CRO utilized an algorithm to randomize the patients into three different arms with comparable demographics—placebo, EZC, or EZC+vitamin D (EZCD) in a double blinded manner. The placebo was composed of rice concentrate.

A total of 180 individuals were enrolled in the placebo control arm. A total of 120 individuals were enrolled in the EZC intervention arm. A total of 60 individuals were enrolled in the EZCD intervention arm. Enrollment was completed during the initial 90 days and the intervention component was carried out over the subsequent six months.

Individuals with the following medical conditions were excluded: ragweed allergy, chronic seasonal allergies, liver disease, autoimmune or connective tissue disorder (e.g., rheumatoid arthritis, lupus, multiples sclerosis, HIV), alcohol consumption more than 7 drinks per week, or more than 3 drinks per occasion, routine recreational drug use, renal disease, and females that were pregnant, wanted to become pregnant for the duration of the study, or who were breastfeeding.

The study was conducted with the CRO's technology platform utilized to screen, enroll and capture study data of the participants. Study enrollees had to actively participate in the study intervention only when they had a URI. Once participants had a URI, they took the double blinded test product as directed and completed the daily symptom survey score until their symptoms resolved.

Participants also recorded any adverse or ill effects any time after taking the test product and for any final adverse events upon completion of the exit form. Participants also recorded if they took any additional medications during the course of their URI.

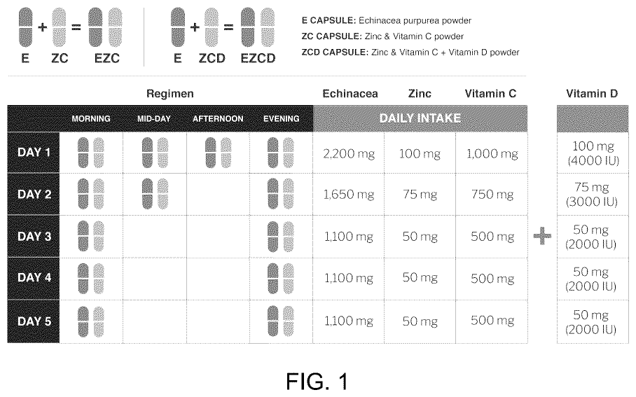

Once a study participant reported the first symptoms of a URI, they were advised to take the intervention (i.e., placebo, EZC, or EZCD) taper dosed over a five-day period. As shown in FIG. 1, the participant was instructed to take the intervention 4 times a day (every 6 hours) on Day 1, 3 times a day (every 8 hours) on Day 2, and 2 times a day (every 12 hours) on Day 3 through Day 5 (see FIG. 1).

### Analysis

In order to evaluate the performance of the intervention, a comparative analysis on the illness of trial participants from the intervention arms EZC and EZCD was performed versus the placebo control arm. There were 360 subjects enrolled in this study, well distributed across the mainland US. The age range across the sample was between 22 years and 88 years old. The average age for the placebo group was 57 years old, the EZC group was 55 years old, and the EZCD group was 54 years old. Most participants were White or Caucasian. The other ethnicities represented less than 10% of the sample within each intervention arm (Table 1).

TABLE 1
Demographic Characteristics of Participants

| | Placebo Group | | EZC Group | | EZC D Group | |
|---|---|---|---|---|---|---|
| Variable | (n = 165) | | (n = 123) | | (n = 72) | |
| Gender n (%) | | | | | | |
| Male | 44 | (26.7) | 28 | (22.8) | 19 | (26.4) |
| Female | 121 | (73.3) | 95 | (77.2) | 53 | (73.6) |
| Age (years) | | | | | | |
| Mean ± SD | 56.9 ± 13.32 | | 54.5 ± 14.83 | | 53.8 ± 14.58 | |
| Median | 59 | | 58 | | 56 | |
| Min, Max | 22, 86 | | 22, 88 | | 23, 78 | |
| Ethnicity n (%) | | | | | | |
| White or Caucasian | 117 | (70.9) | 100 | (81.3) | 60 | (83.3) |
| Black or African | 12 | (7.3) | 6 | (4.9) | 4 | (5.6) |
| American | | | | | | |
| Hispanic or Latino | 14 | (8.5) | 7 | (5.7) | 4 | (5.6) |
| East Asian | 6 | (3.6) | 1 | (0.8) | 1 | (1.4) |
| South Asian/Indian | 6 | (3.6) | 1 | (0.8) | 1 | (1.4) |
| subcontinent | | | | | | |
| Native American or | 2 | (1.2) | 1 | (0.8) | 1 | (1.4) |
| Alaska Native | | | | | | |
| Middle Eastern or | 0 | | 1 | (0.8) | 0 | |
| North African | | | | | | |
| Multiracial or | 7 | (4.2) | 4 | (3.3) | 1 | (1.4) |
| Multiethnic | | | | | | |
| Other | 1 | (0.6) | 2 | (1.6) | 0 | |
| Household income | | | | | | |
| n (%) | | | | | | |
| $30,000 or less | 53 | (32.1) | 22 | (17.9) | 12 | (16.7) |
| $30,000 to $60,000 | 40 | (24.2) | 35 | (28.5) | 23 | (31.9) |
| $60,000 to $100,000 | 31 | (18.8) | 36 | (29.3) | 20 | (27.8) |
| $100,000 to $150,000 | 25 | (15.2) | 19 | (15.4) | 10 | (13.9) |
| $150,000 or more | 16 | (9.7) | 11 | (8.9) | 7 | (9.7) |
| Education level n (%) | | | | | | |
| No degree | 4 | (2.4) | 0 | | 1 | (1.4) |
| High school or | 29 | (17.6) | 27 | (22.0) | 15 | (20.8) |
| equivalent | | | | | | |
| Bachelor's degree | 67 | (40.6) | 49 | (39.8) | 28 | (38.9) |
| Master's degree | 43 | (26.1) | 28 | (22.8) | 13 | (18.1) |
| PhD | 1 | (0.6) | 3 | (2.4) | 3 | (4.2) |
| Other | 21 | (12.7) | 16 | (13.0) | 12 | (16.7) |

Two evaluations were carried out. The primary evaluation assessed illness on the basis of daily symptom and vital sign reporting. Secondarily, we performed an analysis on subjective patient satisfaction. In addition to separate comparisons, EZC and EZCD arms were also combined to include all participants from both pools.

Given that the entire adult population of the United States, or 258.3 million adults, can be infected by URIs, a 90% confidence interval with a 5% margin of error was used to select a 360 patient sample size for this initial feasibility study.

We evaluated the performance of each illness via two metrics: Days To Recovery (DTR) and Symptom Severity During Recovery (SSDR). Two separate, parallel analyses were carried out for each to determine the efficacy of the intervention versus the placebo.

### Days To Recovery (DTR)

DTR is a metric that measures the total number of days in which a patient is experiencing symptoms of an illness while taking the intervention or placebo. DTR also directly lends itself for use in a log-rank analysis, which is used to compute statistical significance.

The primary limitation of DTR arises from the heterogeneous nature of the illness and the patient's unique recovery. Despite aggressive daily oversight, some participants were lost in follow up, most particularly, but perhaps not surprisingly, in the placebo group (Table 2). Interestingly, the 20% dropout rate in the placebo group was comparable to the proportion of patients, approximately 20%, one would expect to have a bacterial infection that may require antibiotics. In these instances of dropout, a DTR score of 14 days was imputed, equivalent to the average number of days a bacterial infection lasts if left untreated.

TABLE 2
Distribution of Subject Completion and Dropout Status by Group

| Group | Target | Completed | Dropouts |
|---|---|---|---|
| EZC | 60 | 58 | 2 |
| EZCD | 32 | 32 | 0 |
| EZC & EZCD | 92 | 90 | 2 |
| Combined | | | |
| Placebo | 90 | 72 | 18 |
| Total | 182 | 162 | 20 |

TABLE 3
Mean DTR Score and Improvement
over Placebo with Log-Rank test

| | Mean | Improvement | p-value |
|---|---|---|---|
| | DTR | Over | (log-rank test; |
| Group | Score | Placebo | versus placebo) |
| EZC | 5.82 | 1.44 days | 0.022 |
| | days | | |
| EZCD | 5.97 | 1.29 days | 0.124 |
| | days | | |
| EZC & EZCD | 5.87 | 1.39 days | 0.017 |
| Combined | days | | |
| Placebo | 7.26 | N/A | N/A |
| | days | | |

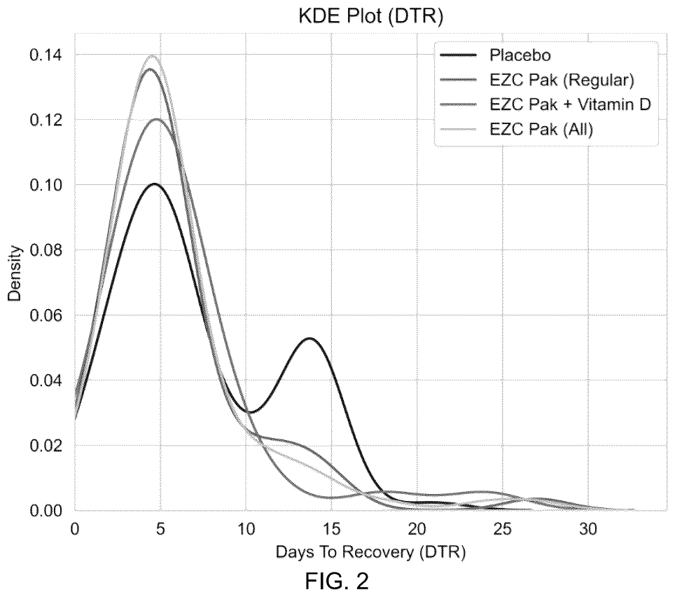

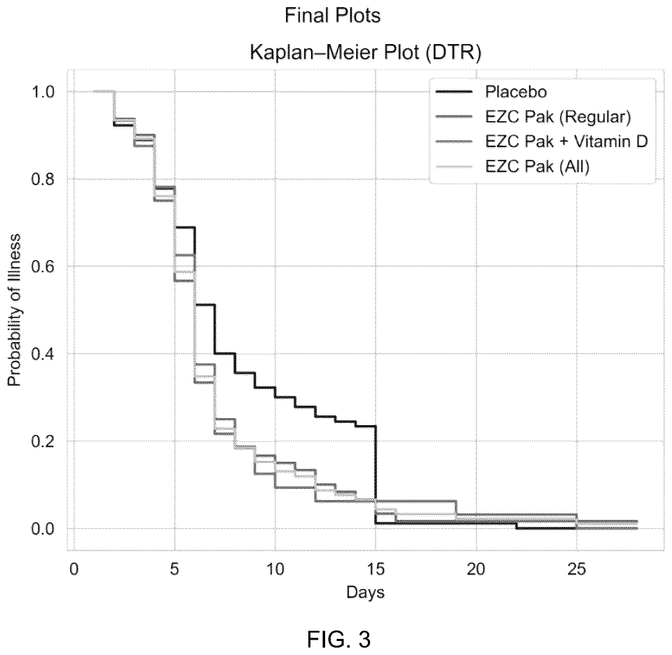

From the numerical results, the average EZC participant recovers nearly one and a half days sooner than the average placebo participant (see Table 3). From the KDE plot and KM plot (see FIGS. 2 and 3), we can similarly observe a higher density of early recoveries as compared to the placebo group, with EZC group peaks coming slightly sooner. For EZC and the EZC Combined cohort, we see that this is a statistically significant improvement (p<0.1). EZCD does not achieve the same level of statistical significance in the DTR analysis driven by the small sample size.

### Symptom Severity During Recovery (SSDR)

SSDR is a metric that aims to capture information on how the symptoms of an illness progress during the recovery phase. Unlike DTR, it is intended to be disease agnostic, where scores range strictly from 0 to 1 regardless of the type and severity of an illness.

SSDR is motivated by the idea that the path to recovery provides useful insights into a participant's disease response. In order to assess this, SSDR is fundamentally based on the area under an illness curve. Such illness curves are generated by measuring daily symptom magnitudes for each illness day, as counted for DTR.
Daily symptom magnitudes utilized the following questions:
Are you experiencing coughing?
Are you experiencing hoarseness?
Are you experiencing runny nose?
Are you experiencing nasal congestion?
Are you experiencing sneezing?
Are you experiencing scratchy or sore throat?
Are you experiencing headache?
Are you experiencing ear pain?
Are you experiencing fatigue?
Are you experiencing chills?
Are you experiencing shortness of breath or difficulty breathing?
Are you experiencing new loss of taste or smell?
Are you experiencing nausea?
Are you experiencing vomiting?
Are you experiencing diarrhea?
Do you have a fever?

Subjective responses for each of 15 symptoms were registered as either “None,” “Mild,” “Moderate,” or “Severe.” Each of these was mapped to a numerical score of 0, 1, 2, and 3 respectively. Additionally, along with the symptoms, the presence of a fever was included, where “No Fever,” “Between 99.9-100.4,” “Between 100.5-101.4,” “Between 101.5-103,” and “More than 103” was similarly mapped to 0, 1, 2, and 3, respectively.

The total “symptom severity” for a day was the sum of these numerical scores across all symptoms and temperature. Other vitals including respiratory rate, heart rate, blood pressure, and oxygen saturation were recorded in the study, but ultimately discarded due to the observed high unreliability of participant self-measurement and recording of these vital signs.

From these symptom scores, a sequence of daily symptom severities was generated, the sum of which is the area under the illness curve. In its raw form, the area represents the total discomfort or pain that a patient experiences during an illness. Reducing this is generally a foundational goal.

With respect to illness heterogeneity, simply taking the raw area under the illness curve brings the same challenges as DTR, only to a larger extent. As such, a scaling approach is used where the symptom severity is scaled according to the illness peak, and the timesteps are scaled based on the length of the recovery phase. This is also equivalent to the ratio of the observed area under recovery to the area under recovery if the illness had stayed static and not improved at all from the peak.

In effect, this results in a theoretical maximum SSDR of 1 and a minimum of 0. The lower the SSDR, the better. SSDR utilizes the area under the recovery phase and not the entire illness. To determine the start of recovery, the greatest one-day decrease in symptom severity was identified and then analyzed backwards until symptom severity stopped improving. In effect, this results in SSDR being a metric that focuses on the severity of symptoms.

SSDR can still be difficult to compute under certain circumstances. For example, in instances where a participant skipped multiple days of data entry, the exact shape of the illness curve becomes unknown. To the extent possible, this was addressed using a linear estimate of nearby datapoints, but such estimates become unreliable if an excess number of data entries are missing.

As such, some basic filters were utilized to assess subject participants with usable and ultimately clinically relevant SSDRs:
1. Participants with incomplete end of data entry reporting or more than 30% of their daily data entries missing
2. Illnesses that were less than three days or longer than ten days

This results in a SSDR Cohort:

TABLE 4
Post-Filter SSDR Cohort

| | | | Mean SSDR | Improvement | | p-value (T-test test; |
|---|---|---|---|---|---|---|
| Group | Size | Post Filter | Score | Over Placebo | 90% CI | versus Placebo) |
| EZC Pak | 58 | 40 | 0.35 | 16.38% | 16.0 to 16.8 | 0.054 |
| EZC Pak + D | 32 | 18 | 0.34 | 19.76% | 19.2 to 20.3 | 0.077 |
| EZC Combined | 90 | 58 | 0.35 | 17.43% | 17.1 to 17.8 | 0.029 |
| Placebo | 72 | 42 | 0.42 | N/A | N/A | N/A |

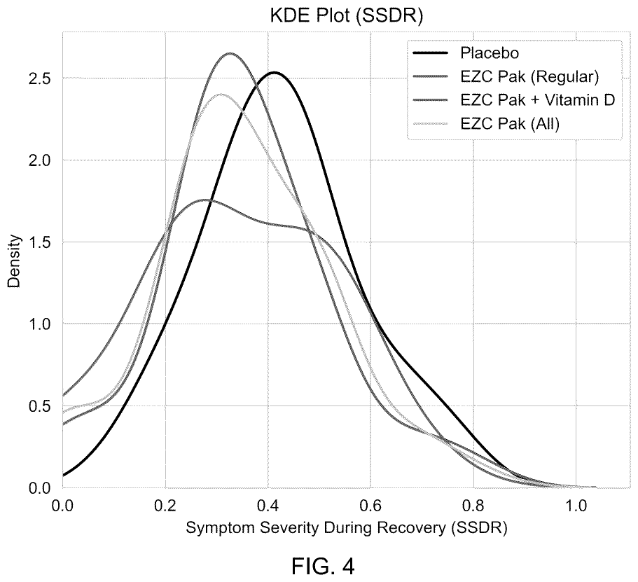

As shown in FIG. 4, there was a 15-20% improvement in SSDR when comparing the mean performance of the EZC groups versus placebo (also see Table 5). In the KDE plot, we also observe the very clear leftward shift in EZC group peaks. Most EZC participants have notably lower SSDRs than their corresponding placebo counterparts.

TABLE 5
Mean SSDR Score and Improvement Over Placebo with t-test

| | | | p-value |
|---|---|---|---|
| | Mean | Improvement | (T-test test; |
| | SSDR | Over | versus |
| Group | Score | Placebo | placebo) |
| EZC | 0.35 | 16.38% | 0.054 |
| EZCD | 0.34 | 19.76% | 0.077 |
| EZC & EZCD | 0.35 | 17.43% | 0.029 |
| Combined | | | |
| Placebo | 0.42 | N/A | N/A |

### Patient Satisfaction Survey Analysis

At the end of illness, participants were asked the following: “Do you believe the test product sped up your recovery time?” The options they could choose were “Yes,” “Maybe,” and “No.” These responses were mapped to scores 1, 0, and −1 respectively, enabling a scale that ranges [−1, 1]. Negative scores imply a lack of confidence in the product, zero implies ambivalence, and positive scores imply satisfaction. No filters were applied or imputations made.

TABLE 6
Mean Satisfaction Score with t-test

| | Mean Satisfaction | p-value (T-test test; |
|---|---|---|
| Group | Score | versus Placebo) |
| EZC Pak | 0.76 | 0.012 |
| EZC Pak + D | 0.30 | 0.893 |
| EZC Pak Combined | 0.63 | 0.051 |
| Placebo | 0.26 | N/A |

All EZC groups had a higher mean participant satisfaction than the placebo group, with the base EZC performing approximately three times better than placebo (see Table 6).

### Patient Safety Data & Side Effects Analysis

In total, 25 participants reported side effects. The most significant side effect was gastrointestinal (GI) discomfort or nausea. This was reported in 6 patients total in the intervention arms, representing 6.5% of the total participants in EZC Combined. This rate of GI discomfort or nausea side effect was within the normal anticipated range given the known potential side effect of high dose zinc.

Three patients in the placebo group reported loose stool or diarrhea. Rice concentrate can have an osmotic effect, especially if taken without additional food. In all but two cases, only a single side effect was reported. One placebo participant reported nausea as well as muscle aches. Another placebo participant reported headache and drowsiness (see Table 7).

TABLE 7
Side Effects Table

| | Placebo | EZC | EZC D | EZC |
|---|---|---|---|---|
| | Group | Group | Group | Combined |
| Side Effect n (%) | (n = 90) | (n = 60) | (n = 32) | (n = 92) |
| GI Discomfort/Nausea | 2 (2.2) | 5 (8.3) | 1 (3.1) | 6 (6.5) |
| Vomiting | 0 | 1 (1.7) | 0 | 1 (1.1) |
| Loose Stool/Diarrhea | 3 (3.3) | 1 (1.7) | 0 | 1 (1.1) |
| Dizziness | 0 | 1 (1.7) | 0 | 1 (1.1) |
| Headache | 1 (1.1) | 0 | 0 | 0 |
| Drowsiness | 1 (1.1) | 1 (1.7) | 0 | 1 (1.1) |
| Insomnia | 1 (1.1) | 0 | 0 | 0 |
| Mouth Dryness | 1 (1.1) | 0 | 0 | 0 |
| Nasal Dryness | 0 | 1 (1.7) | 0 | 1 (1.1) |
| Facial Flushing | 0 | 0 | 1 (3.1) | 1 (1.1) |
| Itching | 1 (1.1) | 0 | 0 | 0 |
| Muscle Ache | 2 (2.2) | 0 | 0 | 0 |

The results of this study of EZC's potential impact on outpatient URI management yield three intriguing results. One, initiating use of EZC as the first step in patients with non-specific URI symptoms has the potential to reduce the duration of illness. The findings support the use of EZC as a viable alternative to patient request for antibiotics when none are deemed clinically necessary at the time of initial clinical presentation. The benefit of EZC on illness duration is likely highest in viral URI. Whether there is a synergistic benefit to using EZC in combination with an antibiotic when a bacterial URI is suspected can be investigated further in future studies.

Secondly, there is a clinically meaningful reduction in symptom severity during URI when using EZC. This lends clinical management to consider EZC alone or in combination with adjuvant treatments that can potentially provide a synergistic reduction in symptom severity during URI (e.g., nasal rinse, steam inhalation, or symptom specific relieving medications).

While epidemiologic data suggests patients with low serum vitamin D levels have a higher incidence of URIs, similar to the outcomes of other recent vitamin D trials, the addition of vitamin D during the acute phase of illness in this study neither benefited nor harmed outcomes with respect to illness duration or symptom severity. As such, the decision to replete vitamin D in the acute phase of URI is an individualized decision left to the patient and their clinician.

The patient satisfaction score of EZC compared favorably versus placebo. This highlights an important potential role EZC can play in maintaining or enhancing patient satisfaction in the clinical management of viral URI. This is particularly important given the predominantly self-limited nature of outpatient cases.

EZC can play a critical role in improving outpatient URI management and antibiotic stewardship. Future study can expand the sample size of this initial study to further evaluate these initial key findings.

1. A method of treating a viral infection, comprising:
obtaining an orally deliverable system,
wherein the orally deliverable system comprises:
a powder form of Echinacea purpurea,
zinc, and
vitamin C,
wherein the powder form of Echinacea purpurea is from 1100 mg to 2200 mg,
wherein the zinc is from 50 mg to 100 mg, and
wherein the vitamin C is from 500 mg to 1000 mg; and
orally ingesting the orally deliverable system.

2. The method of claim 1, wherein the orally deliverable system further comprises:
vitamin D, wherein the vitamin D is from 50 mcg to 100 mcg.

3. A method of treating symptoms of an upper respiratory infection, comprising:
obtaining an orally deliverable system,
wherein the orally deliverable system comprises:
a powder form of Echinacea purpurea,
zinc, and
vitamin C,
wherein the powder form of Echinacea purpurea is from 1100 mg to 2200 mg,
wherein the zinc is from 50 mg to 100 mg, and
wherein the vitamin C is from 500 mg to 1000 mg; and
orally ingesting the orally deliverable system.

4. The method of claim 3, wherein the orally deliverable system further comprises:
vitamin D, wherein the vitamin D is from 50 mcg to 100 mcg.

5. The method according to claim 1, wherein the method does not include using antibiotics.

6. The method according to claim 1, wherein a Day To Recovery (DTR) time is less than about 6 days.

7. The method according to claim 1, wherein the zinc is zinc acetate.

8. The method according to claim 1, wherein the zinc is an ionizable form of zinc.

9. The method according to claim 1, wherein the method does not include using antibiotics;
wherein a Day To Recovery (DTR) time is less than about 6 days; and
wherein the zinc is zinc acetate.

10. The method according to claim 1, wherein the method does not include using antibiotics;
wherein a Day To Recovery (DTR) time is less than about 6 days; and
wherein the zinc is an ionizable form of zinc.

11. The method according to claim 2, wherein the method does not include using antibiotics.

12. The method according to claim 2, wherein a Day To Recovery (DTR) time is less than about 6 days.

13. The method according to claim 2, wherein the zinc is zinc acetate.

14. The method according to claim 2, wherein the zinc is an ionizable form of zinc.

15. The method according to claim 3, wherein the method does not include using antibiotics.

16. The method according to claim 3, wherein a Day To Recovery (DTR) time is less than about 6 days.

17. The method according to claim 3, wherein the zinc is zinc acetate.

18. The method according to claim 3, wherein the zinc is an ionizable form of zinc.

19. The method according to claim 3,
wherein the method does not include using antibiotics;
wherein a Day To Recovery (DTR) time is less than about 6 days; and
wherein the zinc is zinc acetate.

20. The method according to claim 3,
wherein the method does not include using antibiotics;
wherein a Day To Recovery (DTR) time is less than about 6 days; and
wherein the zinc is an ionizable form of zinc.
